(19) United States
(12) Patent Application Publication
(10) Pub. No.: US 2006/0211111 A1
(43) Pub. Date: Sep. 21, 2006

(54) Compositions and methods for neural cell production and stabilization

(76) Inventors: Maisam Mitalipova, Athens, GA (US); Ian Lyons, Buffalo, NY (US); Brian G Condie, Athens, GA (US); Allan J Robins, Athens, GA (US); Scott Allen Noggle, New York, NY (US)

Correspondence Address: Sutherland, Asbill & Brennan/Atta: Bill Warren, 999 Peachtree Street, NE, Atlanta, GA, 30309-3996, US

(21) Appl. No.: 10/539,951

(22) PCT Filed: Dec. 18, 2003
(86) PCT No.: PCT/US03/40762

§ 371 Date: Feb. 5, 2006

(30) Foreign Application Priority Data
Dec. 18, 2002 (US) 60434786

(51) Int. Cl. C12N 5/08

(52) U.S. Cl. 435/368

(57) ABSTRACT

The present invention provides compositions and methods for mammalian neural cell production, their stabilization and their proliferation. More particularly, the present invention provides cellular differentiation methods employing culturing the cells on a cell line or in cell culture and further contacting the cells with MEDII conditioned medium for the generation of stable mammalian neural cells from pluripotent mammalian stem cells. The invention further provides methods for the stabilization of a neural cell in culture comprising contacting the neural cell with MEDII conditioned medium. Preferably, the stabilized neural cell is a neural progenitor cell.

### BACKGROUND OF THE INVENTION

1. Field of the Invention

The present invention generally relates to methods of producing, differentiating and culturing neural cells, maintaining, stabilizing and/or proliferating neural cells, the cells created by these methods and their uses thereof.

2. Background Art

Embryonic Stem (ES) cells represent a powerful model system for the investigation of mechanisms underlying pluripotent cell biology and differentiation within the early embryo, as well as providing opportunities for genetic manipulation of mammals and resultant commercial, medical and agricultural applications. Furthermore, appropriate proliferation and differentiation of ES cells can be used to generate an unlimited source of cells suited to transplantation for treatment of diseases that result from cell damage or dysfunction. Other pluripotent cells and cell lines including early primitive ectoderm-like (EPL) cells as described in International Patent Application WO 99/53021, in vivo or in vitro derived ICM/epiblast, in vivo or in vitro derived primitive ectoderm, primordial germ cells (EG cells), teratocarcinoma cells (EC cells), and pluripotent cells derived by dedifferentiation or by nuclear transfer will share some or all of these properties and applications.

The successful isolation, long-term clonal maintenance, genetic manipulation and germ-line transmission of pluripotent cells has generally been difficult and the reasons for this are unknown. International Patent Application WO 97/32033 and U.S. Pat. No. 5,453,357 describe pluripotent cells including cells from species other than rodents. Human ES cells have been described in International Patent Application WO 00/27995, and in U.S. Pat. No. 6200806, and human EG cells have been described in International Patent Application WO 98/43679.

The ability to tightly control differentiation or form homogeneous populations of partially differentiated or terminally differentiated cells by differentiation in vitro of pluripotent cells has proved problematic. Current approaches involve the formation of embryoid bodies from pluripotent cells in a manner that is not controlled and does not result in homogeneous populations. Mixed cell populations such as those in embryoid bodies of this type are generally unlikely to be suitable for therapeutic or commercial use.

Selection procedures have been used to obtain cell populations enriched in neural cells from embryoid bodies. These include manipulation of culture conditions to select for neural cells (Okabe et al., 1996 Dev. Biol. 176:300-312), and genetic modification of ES cells to allow selection of neural cells by antibiotic resistance (Li et al., 1998 Current Biol. 8:971-974). In these procedures, the differentiation of pluripotent cells in vitro does not involve biological molecules that direct differentiation in a controlled manner. One publication (Kawasaki et al., 2002 Proc. Natl. Acad. Sci. USA 99(3):1580-1585) discloses the generation of dopaminergic neurons from primate ES cells by stromal cell-derived activity. However it was not possible to maintain or proliferate the neuronal cells using this method. Hence homogeneous populations of neural cells are difficult to maintain and proliferate, restricting the ability to derive essentially homogeneous populations of partially differentiated or differentiated neural cells.

In summary, it has not been possible to control the differentiation of pluripotent cells in vitro, to provide homogeneous, synchronous populations of neural cells with unrestricted neural differentiation capacity that can be suitably maintained and that will further proliferate. These limitations have restricted the ability to form essentially homogeneous, synchronous populations of partially differentiated and terminally differentiated neural cells in vitro, and have restricted their further development for therapeutic and commercial applications.

Neural stem cells and precursor cells have been derived from fetal brain and adult primary central nervous system tissue in a number of species, including rodent and human (e.g. see U.S. Pat. No. 5,753,506 (Johe), U.S. Pat. No. 5,766,948 (Gage), U.S. Pat. No. 5,589,376 (Anderson and Stemple), U.S. Pat. No. 5,851,832 (Weiss et al.), U.S. Pat. No. 5,958,767 (Snyder et al.), WO99/32606 (Brustle) and U.S. Pat. No. 5,968,829 (Carpenter)). However, each of these disclosures fails to describe a predominantly homogeneous population of neural stem cells able to differentiate into all neural cell types of the central and peripheral nervous systems, and/or essentially homogeneous populations of partially differentiated or terminally differentiated neural cells derived from neural stem cells by controlled differentiation and/or cells that are fated Furthermore, it is not clear whether cells derived from primary fetal or adult tissue can be expanded sufficiently to meet potential cell and gene therapy demands.

There is a need, therefore, to identify methods and compositions for the production of a population of cells enriched in neural stem cells, the maintenance or stabilization, and proliferation of these cells, and the products of their further differentiation.

### SUMMARY OF THE INVENTION

It is an object of the present invention to overcome, or at least alleviate, one or more of the difficulties or deficiencies associated with the prior art. In that regard, the present invention provides a method of stabilizing a neural cell comprising: (a) providing a pluripotent mammalian cell; (b) culturing the pluripotent mammalian cell to produce a neural cell; and (c) contacting the neural cell with a MEDII conditioned medium for greater than 2 passages to thereby stabilize the neural cell. The present invention also provides a method of enriching a culture of neural cells using the method above. In a further embodiment of the claimed method, step (b) comprises the use of a medium comprising a compound selected from the group consisting of KSR, GMEM, HES medium, or any combination thereof.

In a further embodiment of the above-method, step (b) occurs in the presence of a feeder cell layer. In one embodiment, the feeder cell layer comprises a stromal cell. In further embodiments, the stromal cell is a murine stromal cell. Preferably, the murine stromal cell is a PA6 cell. Alternatively, the stromal cell is a human stromal cell. Other feeder cell layers may be used as known in the art. In other embodiments, step (b) of the above-method occurs in the absence of a feeder cell layer.

The present invention further provides a method of stabilizing a neural cell comprising: (a) providing a mammalian neural cell; and (b) contacting the neural cell with a MEDII conditioned medium for greater than 2 passages to thereby stabilize the neural cell.

The present invention contemplates that the methods can comprise an additional step of contacting the neural cell with a differentiating medium prior to contact with the MEDII conditioned medium. In one embodiment, the differentiating medium is essentially serum free. In a further embodiment, the differentiating medium comprises a base salt solution, wherein the base salt solution is selected from the group consisting of DMEM, GMEM, or any combination thereof. In other embodiments, the differentiating medium further comprises supplements selected from the group consisting of N2, FGF2, or any combination thereof.

The pluripotent mammalian cell of the present invention can be selected from, but is not limited to, the group consisting of an embryonic stem cell; an ICM/epiblast cell; a primitive ectoderm cell; a primordial germ cell; and a teratocarcinoma cell. The pluripotent mammalian cell is preferably human but it is to be understood the invention may apply to any mammalian species including murine or primate derived cells. In one embodiment, the mammalian pluripotent cell is a human embryonic stem cell. In preferred embodiments, the stabilized neural cell produced by the methods of the present invention is a neural progenitor cell. Preferably the neural progenitor cell is a human neural progenitor cell.

In a preferred embodiment, the MEDII conditioned medium is an essentially serum free medium. In a further embodiment, the MEDII conditioned medium is optionally essentially LIF free. The present invention contemplates that the MEDII conditioned medium is a HepG2 conditioned medium. Alternatively, the MEDII conditioned medium can comprise a biologically active component selected from the group consisting of: (a) a large molecular weight extracellular matrix protein; (b) a low molecular weight component comprising proline; (c) a biologically active fragment of any of the proteins or components described in a) or b); (d) an analog of any of the proteins or components described in a) or b); (e) a neural inducing factor; and (f) any combination thereof. In one embodiment, the MEDII conditioned medium comprises a large molecular weight extracellular matrix protein.

In certain embodiments of the present invention, the neural cell is plated prior to contact with the MEDII conditioned medium. In one embodiment, the neural cell is plated with a feeder cell layer prior to contact with the MEDII conditioned medium. In another embodiment, the neural cell is plated on a substrate prior to contact with the MEDII conditioned medium.

In certain embodiments, the neural cell is isolated prior to contacting the neural cell with the MEDII conditioned medium. Preferably, isolating the neural cell comprises manually selecting the neural cell based at least partially on its morphology.

The present invention contemplates that the stabilized neural cell generated by the present invention can be further differentiated to produce a differentiated neural cell. In one embodiment, the differentiated neural cell is TH positive, and expresses DAT and V-MAT. In another embodiment, a population of differentiated neural cells is produced, and at least 50% of the population of differentiated neural cells is TH positive. In another embodiment, the differentiated neural cell expresses glutamate decarboxylase. In a further embodiment, the differentiated neural cell expresses GFAP.

The invention further contemplates a composition comprising an isolated neural cell, wherein the cell expresses nestin, wherein the cell has been stabilized by contact with MEDII conditioned medium for greater than 2 passages, and wherein the cell can differentiate into more than one type of further differentiated neural cell. In other embodiments, the cell has been stabilized by contact with MEDII conditioned medium for greater than 5 passages, greater than 10 passages, greater than 20 passages, greater than 30 passages, or for longer than approximately 1 year. Preferably the neural cell is a neural progenitor cell.

### DETAILED DESCRIPTION OF THE INVENTION

Applicant has demonstrated that culturing pluripotent mammalian cells on a cell line or a culturing medium to generate a partially differentiated neural cell, and then contacting the neural cells with MEDII conditioned medium results in the prolonged stabilization of the neural cell in culture in comparison to a neural cell cultured without the addition of the MEDII conditioned medium.

In that regard, the present invention provides a method of stabilizing a neural cell comprising: (a) providing a pluripotent mammalian cell; (b) culturing the pluripotent mammalian cell to produce a neural cell; and (c) contacting the neural cell with a MEDII conditioned medium for greater than 2 passages to thereby stabilize the neural cell. The present invention also provides a method of enriching a culture of neural cells using the method above. In a further embodiment of the claimed method, step (b) comprises the use of a medium comprising a compound selected from the group consisting of KSR, GMEM, HES medium, or any combination thereof.

In a further embodiment of the above-method, step (b) occurs in the presence of a feeder cell layer. In one embodiment, the feeder cell layer comprises a stromal cell. In further embodiments, the stromal cell is a murine stromal cell. Preferably, the murine stromal cell is a PA6 cell. Alternatively, the stromal cell is a human stromal cell. Other feeder cell layers may be used as known in the art, such as mouse embryonic fibroblasts (MEFs), or other human cell lines that may substitute for MEFs. In one embodiment, the neural cell is produced from the pluripotent cell without the formation of an embryoid body. In other embodiments, the neural cell is produced in the absence of a feeder cell layer.

The present invention further provides a method of stabilizing a neural cell comprising: (a) providing a mammalian neural cell; and (b) contacting the neural cell with a MEDII conditioned medium for greater than 2 passages to thereby stabilize the neural cell.

The invention further contemplates a composition comprising an isolated neural cell, wherein the cell expresses nestin, wherein the cell has been stabilized by contact with MEDII conditioned medium for greater than 2 passages, and wherein the cell can differentiate into more than one type of further differentiated neural cell. In other embodiments, the cell has been stabilized by contact with MEDII conditioned medium for greater than 5 passages, greater than 10 passages, greater than 20 passages, greater than 30 passages, or for longer than approximately 1 year. Preferably the neural cell is a neural progenitor cell.

The present invention contemplates that the methods can comprise an additional step of contacting the neural cell with a differentiating medium prior to contact with the MEDII conditioned medium. In one embodiment, the differentiating medium is essentially serum free. In a further embodiment, the differentiating medium comprises a base salt solution, wherein the base salt solution is selected from the group consisting of DMEM, GMEM or any combination thereof. In other embodiments, the differentiating medium further comprises supplements selected from the group consisting of N2, FGF2, or any combination thereof.

The pluripotent mammalian cell of the present invention can be selected from, but is not limited to, the group consisting of an embryonic stem cell; an ICM/epiblast cell; a primitive ectoderm cell; a primordial germ cell; and a teratocarcinoma cell. The pluripotent mammalian cell is preferably human but it is to be understood the invention may apply to any mammalian species including murine or primate derived cells. In one embodiment, the mammalian pluripotent cell is a human embryonic stem cell. In preferred embodiments, the stabilized neural cell produced by the methods of the present invention is a neural progenitor cell. Preferably the neural progenitor cell is a human neural progenitor cell.

In a preferred embodiment, the MEDII conditioned medium is an essentially serum free medium. In a further embodiment, the MEDII conditioned medium is optionally essentially LIF free. The present invention contemplates that the MEDII conditioned medium is a HepG2 conditioned medium. Alternatively, the MEDII conditioned medium can comprise a biologically active component selected from the group consisting of: (a) a large molecular weight extracellular matrix protein; (b) a low molecular weight component comprising proline; (c) a biologically active fragment of any of the proteins or components described in a) or b); (d) an analog of any of the proteins or components described in a) or b); (e) a neural inducing factor; and (f) any combination thereof In one embodiment, the MEDII conditioned medium comprises a large molecular weight extracellular matrix protein.

In certain embodiments of the present invention, the neural cell is plated prior to contact with the MEDII conditioned medium. In one embodiment, the neural cell is plated with a feeder cell layer prior to contact with the MEDII conditioned medium. In another embodiment, the neural cell is plated on a substrate prior to contact with the MEDII conditioned medium.

In certain embodiments, the neural cell is isolated prior to contacting the neural cell with the MEDII conditioned medium. Preferably, isolating the neural cell comprises manually selecting the neural cell based at least partially upon its morphology.

The present invention contemplates that the stabilized neural cell generated by the present invention can be further differentiated to produce a differentiated neural cell. In one embodiment, the differentiated neural cell is TH positive, and expresses DAT and V-MAT. In another embodiment, a population of differentiated neural cells is produced, and at least 50% of the population of differentiated neural cells is TH positive. In another embodiment, the differentiated neural cell expresses glutamate decarboxylase. In a further embodiment, the differentiated neural cell expresses GFAP. However, the formation of other neural cell types from the stabilized neural cell is contemplated.

In one embodiment of the invention the pluripotent cells are human ES (hES) cells. These cells are cultured on PA6 feeder cells or MEFs for about 16 days. The media may be hES media, GMEM and 10% KSR or any other suitable media. Alternatively, the cells are cultured in the absence of a feeder cell layer. After about 16 days the cells are further cultured for about 17 days in a differentiating media that includes DMEM/F12 and FGF2 and optionally N2, or any other suitable media. It is preferred that this differentiating medium is essentially serum free, however, some minor amounts of serum may be added at the latter stages. After this, at about 33 days, the cells have formed rosettes. These rosettes are plated onto polyornithine and laminin coated dishes and the cells allowed to proliferate in a conditioned medium containing about 50% MEDII and 50% of a differentiating medium. It was observed that without MEDII the cells were more inclined to spontaneously differentiate and the cells could not be maintained beyond 2 passages. However with MEDII it was observed that a greater number of rosettes formed with greater complexity. A benefit of this method is that the neural cells could be passaged at least ten times, and optimally greater than 30 times, for a period of greater than 1 year, suggesting that the cells may be immortal and capable of being frozen and thawed while retaining the stable characteristics. In addition, a high proportion of the neural cells produced appeared to be fated towards dopaminergic neurons, the cells being TH+, and expressing a dopamine transporter (DAT) and V-MAT, however, this does not preclude the utility of this method for generation of other cell types. It was observed that the neural cells stabilized by the current methods could be differentiated into other neural cell types, such as, but not limited to, glial cells and GABAergic neurons.

It is to be understood that as used in the specification and in the claims, “a” or “an” can mean one or more, depending upon the context in which it is used. Thus, for example, reference to “a cell” can mean that at least one cell can be utilized. As also used herein, the term “MEDII conditioned medium” refers to a medium containing one or more biologically active components as described herein. In a preferred embodiment, the biologically active component is derived from a hepatic or hepatoma cell or cell line culture supernatant. The hepatic or hepatoma cell or cell line can be from any species, however, preferred cell lines are mammalian or avian in origin. The hepatic or hepatoma cell line can be selected from, but is not limited to, the group consisting of: a human hepatocellular carcinoma cell line such as a HepG2 cell line (ATCC HB-8065) or Hepa-1c1c-7 cells (ATCC CRL-2026); a primary embryonic mouse liver cell line; a primary adult mouse liver cell line; a primary chicken liver cell line; and an extraembryonic endodermal cell line such as END-2 and PYS-2. A particularly preferred cell line is the HepG2 cell line (ATCC HB-8065). A description of the isolation of a MEDII medium from a HepG2 cell line is provided in Example 1 below. In one embodiment of the present invention, the MEDII medium is derived from a HepG2 cell line and contains supplements of FGF2 and optionally hLIF.

As used herein, the terms “biologically active component” or “bioactive component” and “bioactive factor” refer to any compound or molecule that induces a pluripotent or multipotent cell to follow a differentiation pathway toward an EPL cell or a neural cell. A bioactive component from the conditioned medium may be used in place of the MEDII conditioned medium in any embodiment described herein. While the bioactive component may be as described below, the term is not limited thereto. The term “bioactive component” as used herein includes within its scope a natural or synthetic molecule or molecules which exhibit(s) similar biological activity.

As used herein when referring to a cell, cell line, cell culture or population of cells, the term “isolated” refers being substantially separated from the natural source of the cells such that the cell, cell line, cell culture, or population of cells are capable of being cultured in vitro. In addition, the term “isolating” is used to refer to the physical selection of one or more cells out of a group of two or more cells, wherein the cells are selected based on cell morphology and/or the expression of various markers.

As used herein, the term “express” refers to the transcription of a polynucleotide or translation of a polypeptide in a cell, such that levels of the molecule are measurably higher in a cell that expresses the molecule than they are in a cell that does not express the molecule. Methods to measure the expression of a molecule are well known to those of ordinary skill in the art, and include without limitation, Northern blotting, RT-PCT, in situ hybridization, Western blotting, and immunostaining.

As used herein, the term “contacting” (i.e., contacting a cell e.g. a pluripotent cell or a neural, with an compound) is intended to include incubating the compound and the cell together in vitro (e.g., adding the compound to cells in culture). The term “contacting” is not intended to include the in vivo exposure of cells to a bioactive component of the MEDII conditioned medium that may occur naturally in a subject (i.e., exposure that may occur as a result of a natural physiological process). The step of contacting the cell with the MEDII conditioned medium can be conducted in any suitable manner. For example, the cells may be treated in adherent culture, or in suspension culture. It is understood that the cells treated with the MEDII conditioned medium may be further treated with other cell differentiation environments to maintain the cells, or to differentiate the cells further. In one embodiment, the cells treated with the compound are further differentiated into neural cells, which can be partially or terminally differentiated neural cells.

Additionally, the term “neural cell” includes, but is not limited to, neurectoderm cells; glial cells; neural cells of the central nervous system such as a dopaminergic cell, differentiated or undifferentiated astrocytes or an oligodendrocytes, neural stem cells, neuronal progenitors, glial progenitors, differentiated neurons such as dopaminergic neurons, and a neural cell of the peripheral nervous system. As used herein, the term “neurectoderm” refers to undifferentiated neural progenitor cells substantially equivalent to cell populations comprising the neural plate and/or neural tube. Neurectoderm cells are multipotent. Therefore, the use of the term “neural cell” in the context of the present invention means that the cell is at least more differentiated towards a neural cell type than the pluripotent cell from which it is derived. Also as used herein, producing a neural cell encompasses the production of a cell culture that is enriched for neural cells. In preferred embodiments, the term “enriched” refers to a cell culture that contains more than approximately 50%, 55%, 60%, 65%, 70%, 75%, 80%, 85%, 90%, or 95% of the desired cell lineage.

The neural cell types that differentiate from embryonic stem cells have several uses in various fields of research and development including but not limited to drug discovery, drug development and testing, toxicology as well as basic science research. These useful neural cell types include neurons of a wide variety of morphologies and phenotypes as well as various types of glial cells such as astrocytes and oligodendrocytes. These cell types express molecules that are of interest in a wide range of research fields. These include the molecules known to be required for the fimctioning of neural cell types as described in standard reference texts and current reviews on neurobiology and neurophysiology (Cooper et al., 1996 In The Biochemical Basis of Neuropharmacology. New York, Oxford University Press; Fain, 1999 In Molecular and Cellular Physiology of Neurons. Cambridge, Mass., Harvard University Press; Kandel et al., 2000 In Principles of Neural Science. New York, McGraw-Hill.; Khakh 2001, Nat. Rev. Neurosci., 2(3):165-74; Bowery et al. 2002 Pharmacol Rev, 54(2):247-64; Howlett et al., 2002 Pharmacol Rev., 54(2):161-202; Laube et al., 2002 Trends Pharmacol. Sci., 23(11):519-27). These molecules include but are not limited to cytokines, growth factors, neurotrophic factors, neuroactive peptides (neuropeptides), cytokine receptors, growth factor receptors, ionotropic and metabotropic neurotransmitter receptors, neurotransmitter transporters including plasma membrane reuptake transporters as well as vesicular neurotransmitter transporters, voltage gated ion channels, and ion pumps. The neural cells also express the enzymes in the biochemical pathways that produce and degrade various neurotransmitters.

The invention provides a composition comprising stabilized neural cells, wherein the neural cells are preferably neural progenitor cells. The neural progenitor cells are characterized by the expression of nestin and/or Musashi-1, and their capacity to differentiate into cells of the neural lineage including neurons and glia. The neural cell types produced by the neural progenitors may include cells of the central or peripheral nervous system, including, but not limited to neurons, astrocytes, oligodendrocytes and Schwann cells. Neuron cell types produced in these cultures may express one or more neurotransmitter phentotypes. These include GABAergic neurons that express glutamate decarboxylase (GAD) or vesicular inhibitory amino acid transporter/vesicular gaba transporter (Viaat/Vgat); cholinergic neurons that express choline acetyltransferase (ChAT/CAT) or vesicular acetylcholine transporter (VAChT); glutamatergic neurons that express the vesicular glutamate transporter; glycinergic neurons that express the vesicular inhibitory amino acid transporter (Viaat/Vgat), noradrenergic neurons that express the norepinephrine transporter (NET); adrenergic neurons that express phenylmethanolamine N-methyl transferase (PNMT); serotonergic neurons that express tryptophan hydroxylase (TrH) or serotonin transporter (SERT); or histaminergic neurons that express histidine decarboxylase (HDC).

Multiple families of signaling molecules can be expressed and functional in neural cells derived from embryonic stem cells. Among the signaling molecules expressed are neurotrophic factors that promote the survival and function of neural cell types. These include but are not limited to nerve growth factor (NGF), brain derived neurotrophic factor (BDNF), neurotrophin-3 (NT-3), neurotrophin-4/5 (NT-4/5), interleukin-6 (IL-6), ciliary neurotrophic factor (CNTF), leukemia inhibitory factor (LIF), cardiotrophin, members of the transforming growth factor (TGF)/bone morphogenetic protein (BMP)/growth and differentiation factor (GDF) family, the fibroblast growth factor family, the glial derived neurotrophic factor (GDNF) family including but not limited to GDNF, neurturin, neublastin/artemin, and persephin and factors related to and including hepatocyte growth factor. In addition, the cells express neurotrophic receptors that can be activated by these neurotrophic factors. These receptors include but are not limited to trk A, trk B, trk C, p75NTR, gp 130, leukemia inhibitory factor receptors, ciliary neurotrophic factor receptors, c-ret receptor tyrosine kinase, the GDNF family receptor family-α receptors GFRα1, GFRα2, GFRα3, GFRα4, the fibroblast growth factor receptors FGFR1, FGFR2, FGFR3, FGFR4, and the type I and type II receptors for the transforming growth factor (TGF)/bone morphogenetic protein (BMP), growth and differentiation factor (GDF) family. The neurotrophin GDNF is currently being tested for its ability to promote the survival of dopaminergic neurons in Parkinson's disease patients. The embryonic stem cell derived neural cells may be a unique system in which to develop therapies based on GDNF infusion or to find small molecules that substitute for the activity of GDNF and GDNF family molecules.

Neural cells derived from embryonic stem cells may express multiple classes of ion channels. These include calcium channels including but not limited to N-type channels blocked by ω-conotoxin-GVIA, P-type channels blocked by ω-Aga-IVA, Q-type channels blocked by ω-conotoxin-MVIIC, R-type channels, T-type channels, L-type channels blocked by DHP. The neural cells may also express chloride channels, sodium channels and potassium channels. The sodium channels include but are not limited to tetrodotoxin sensitive and insensitive types. The potassium channels include but are not limited to channels blocked by tetraethylamonium or 4-aminopyridine and can include but are not limited to delayed rectifier, rapidly inactivating, intermediately inactivating (D-current) potassium channels or inward rectifier potassium channels. The neural cells may also express calcium activated potassium channels including but not limited to BK channels that can be blocked by charybdotoxin or iberiotoxin and SK channels that can be blocked by apamin.

The embryonic stem cell derived neural cells may express ion pumps and ion transporters commonly required for neural cell function. These include the sodium-potassium ATPase α and β subunits. Calcium pumps including the calcium ATPase and the sodium/calcium countertransporter also known as the sodium/calcium exchanger or antiporter. Chloride pumps including the potassium/chloride cotransporter and the sodium/potassium/chloride cotransporter. Hydrogen ion pumps including the sodium/hydrogen ion pump and the anion exchanger protein can also be expressed.

The embryonic stem cell derived neural cells may express the components of gap junctions. These include the connexin proteins. Expression of gap junctions will lead to the formation of electrical synapses between the neural cells providing a means to study this aspect of intercellular communication among neural cell types.

Multiple neurotransmitter receptor types may be expressed by the neural cells produced from embryonic stem cells. These include ionotropic and metabotropic receptors. The receptors for GABA including but not limited to ionotropic GABA_{A} and GABA_{C} receptors and metabotropic GABA_{B} receptors; receptors for acetylcholine including but not limited to the ionotropic nicotinic acetylcholine receptors and metabotropic muscarinic acetylcholine receptors; receptors for glutamate including ionotropic AMPA, kainate and NMDA subtypes and metabotropic glutamate receptors including the group I (mGluR1, mGluR5), group II (mGlu R2, mGluR3), and group III (mGluR 4, 6, 7, 8) receptors; ionotropic glycine receptors including GlyRα1, GlyRα2, GlyRα3, GlyR α4 and GlyRβ; metabotropic cannabinoid receptors including CB1 and CB2, metabotropic dopamine receptors including D1, D2, D3, D4 and D5; metabotropic serotonin (5-hydroxytryptamine) receptors including 5HT1, 5HT2, 5HT4, 5HT5, 5HT6 and 5HT7 and the ionotropic serotonin receptor 5HT3; metabotropic adrenergic receptors including α1, α2, β1, β2, β3; metabotropic histamine receptors including H1, H2 and H3; and ionotropic purinoreceptors including the P2X receptor and metabotropic purinoreceptors including P2Y.

Neural cells derived from embryonic stem cells may express the enzymes for the synthesis and degradation of neurotransmitters. These enzymes include but are not limited to the glutamate decarboxylases (Gad65, Gad67) that synthesize γ-amino butyric acid (GABA) and GABA transaminase the enzyme that degrades GABA. The cells can express tyrosine hydroxylase and aromatic amino acid decarboxylase (dopa decarboxylase) for the biosyntheis of dopamine and dopa-β hydroxylase which is required to convert dopamine into norepinephrine as well as phenylethanolamine-N-methyl transferase which is required to convert norepinephrine to epinephrine. The cells may also express monoamine oxidase and catechol-O-methyltransferase, which degrade dopamine, norepinephrine and epinephrine. The pathway for serotonin (5-hydroxytryptamine) synthesis which consists of the enzymes tryptophan hydroxylase and aromatic amino acid decarboxylase and the pathway for serotonin degradation via monoamine oxidase can be expressed by the embryonic stem cell derived neural cells. The neural cells may express the pathway for acetylchlone synthesis and degradation including the enzymes choline acetyltransferase and acetylcholine esterases. The cells may also express the pathway for histamine synthesis and degradation including histidine decarboxylase, histamine methyl transferase and diamine oxidase (histaminase). Changes in the activity of these enzymes can alter the levels of neurotransmitter within the cell. Drugs that alter the activity of these enzymes can change neurotransmitter levels within cells, thereby altering neurotransmitter function. An example of such a drug is vigabatrin, which inhibits GABA transaminase increasing GABA levels. Vigabatrin is used to treat epilepsy. In addition to these classical neurotransmitters the neural cells will express various neuropeptides that can also act as neurotransmitters.

The neural cells derived from embryonic stem cells may also express neurotransmitter re-uptake transporters (plasma membrane transporters). These molecules are important targets for compounds designed to modulate neurotransmission. Drugs that modulate the re-uptake of serotonin (5-hydroxytryptamine), dopamine, norepinephrine, and GABA are currently in clinical use. The neurotransmitter re-uptake transporters expressed by embryonic stem cell derived neural cells may include, but are not limited to, the choline transporter, dopamine transporter (DAT), serotonin transporter (SERT), norepinephrine transporters (NETs), GABA transporters (GATs), glutamate transporters and glycine transporters (GLYT-1, GLYT-2).

The neural cells derived from embryonic stem cells may express vesicular transporters required for transport of neurotransmitters into synaptic vesicles. These include the vesicular monoamine transporters (VMAT1, VMAT2), the vesicular inhibitory amino acid transporter/vesicular GABA transporter (VIAAT/VGAT), the vesicular glutamate transporter (VGLUT1, VGLUT2, VGLUT3), and the vesicular acetylcholine transporter (VAChT). Compounds altering the function of these vesicular transporters can affect neurotransmission making these transporters potential drug targets.

In a preferred embodiment the neural cells that are stabilized are neural progenitor cells. As used herein, the term “stabilize” refers to the differentiation state of a cell. When a cell or cell population is stabilized, it will continue to proliferate over multiple passages in culture, and preferably indefinitely in culture; additionally, each cell in the culture is preferably of the same differentiation state, and when the cells divide, typically yield cells of the same cell type or yield cells of the same differentiation state. Preferably, a stabilized cell or cell population does not further differentiate or de-differentiate if the cell culture conditions are not altered, and the cells continue to be passaged and are not overgrown. In a preferred embodiment of the present invention, the cell or cell population that is stabilized is a neural cell or cell population. In another preferred embodiment of the present invention, the neural cell or cell population that is stabilized is a neural progenitor cell or cell population. Preferably the neural cell that is stabilized is capable of proliferation in the stable state indefinitely, or for at least more than 2 passages. Preferably, it is stable for more than 5 passages, more than 10 passages, more than 15 passages, more than 20 passages, more than 25 passages, or most preferably, it is stable for more than 30 passages. In one embodiment, the neural cell is stable for greater than 1 year of continuous passaging.

The MEDII conditioned medium described herein can comprise one or more biologically active components selected from the group consisting of a large molecular weight extracellular matrix protein; a low molecular weight component comprising proline; a biologically active fragment of any of the aforementioned proteins or components; and an analog of any of the aforementioned proteins or components. The large molecular weight extracellular matrix protein preferably has a molecular weight of greater than approximately, 10 kD, more preferably between approximately 100-500 kD and most preferably between approximately 210-250 kDa as measured on a 10% reducing/denaturing polyacrylamide gel. In a further preferred embodiment, the large molecular weight extracellular matrix protein comprises a cellular fibronectin protein or a laminin protein. In addition the MEDII conditioned medium may contain a neural inducing factor.

The low molecular weight component of the MEDII conditioned medium can comprise one or more proline residues or a polypeptide containing proline residues. As used herein, the term “polypeptide” refers to any of various amides that are derived from two or more amino acids by combination of the amino group of one acid with the carboxyl group of another and usually obtained by partial hydrolysis of proteins. In a preferred embodiment, the low molecular weight component is L-proline or a polypeptide including L-proline. The proline containing polypeptide preferably has a molecular weight of less than approximately 5 kD, more preferably less than approximately 3 kD. In a further preferred embodiment, the low molecular weight component is a polypeptide of between approximately 2-11 amino acids, more preferably of between approximately 2-7 amino acids and most preferably approximately 4 amino acids. The proline containing polypeptide can be selected from, but is not limited to, the following polypeptides: Pro-Ala, Ala-Pro, Ala-Pro-Gly, Pro-OH-Pro, Pro-Gly, Gly-Pro, Gly-Pro-Ala, Gly-Pro-Ala, Gly-Pro-OH-Pro, Gly-Pro-Arg-Pro (SEQ ID NO:1), Gly-Pro-Gly-Gly (SEQ ID NO:2), Val-Ala-Pro-Gly (SEQ ID NO:3), Arg-Pro-Lys-Pro (SEQ ID NO:4), and Arg-Pro-Lys-Pro-Gln-Gln-Phe-Phe-Gly-Leu-MetOH (SEQ ID NO:5).

While MEDII conditioned medium is proposed for use in the stabilization of neural cells, it is also contemplated that MEDII conditioned medium can be used at any or all stages of derivation of the neural cell from a pluripotent cell.

As used herein “essentially LIF free” refers to a medium that does not contain leukemia inhibitory factor (LIF), or that contains essentially no LIF. “Essentially” means that a de minimus or reduced amount of a component, such as LIF, may be present that does not eliminate the improved bioactive neural cell culturing capacity of the medium or environment. For example, essentially LIF free medium or environment can contain less than approximately 100, 75, 50, 40, 30, 10, 5, 4, 3, 2, or 1 ng/ml LIF, wherein the presently improved bioactive neural cell culturing capacity of the medium or environment is still observed. As used herein, the term “LIF” refers to leukemia inhibitory factor that is obtained or derived from any species, and is therefore not limited to human LIF. In certain embodiments of the above methods, the MEDII conditioned medium is essentially free from LIF.

In a preferred embodiment, the pluripotent cell is a human cell. As used herein, the term “pluripotent human cell” encompasses pluripotent cells obtained from human embryos, fetuses or adult tissues. In one preferred embodiment, the pluripotent human cell is a human pluripotent embryonic stem cell. In another embodiment the pluripotent human cell is a human pluripotent fetal stem cell, such as a primordial germ cell. In another embodiment the pluripotent human cell is a human pluripotent adult stem cell. As used herein, the term “pluripotent” refers to a cell capable of at least developing into one of ectodermal, endodermal and mesodermal cells. As used herein the term “pluripotent” refers to cells that are totipotent and multipotent. As used herein, the term “totipotent cell” refers to a cell capable of developing into all lineages of cells. The term “multipotent” refers to a cell that is not terminally differentiated. As also used herein, the term “multipotent” refers to a cell that, without manipulation (i.e., nuclear transfer or dedifferentiation inducement), is incapable of forming differentiated cell types derived from all three germ layers (mesoderm, ectoderm and endoderm), or in other words, is a cell that is partially differentiated. The pluripotent human cell can be selected from the group consisting of a human embryonic stem (ES) cell; a human inner cell mass (ICM)/epiblast cell; a human primitive ectoderm cell, such as an early primitive ectoderm cell (EPL); a human primordial germ (EG) cell; and a human teratocarcinoma (EC) cell. The human pluripotent cells of the present invention can be derived using any method known to those of skill in the art. For example, the human pluripotent cells can be produced using de-differentiation and nuclear transfer methods. Additionally, the human ICM/epiblast cell or the primitive ectoderm cell used in the present invention can be derived in vivo or in vitro. EPL cells may be generated in adherent culture or as cell aggregates in suspension culture, as described in WO 99/53021. Furthermore, the human pluripotent cells can be passaged using any method known of those to skill in the art, including, manual passaging methods, and bulk passaging methods such as antibody selection and protease passaging.

It is preferable that the MEDII conditioned medium contains no or very little serum. As used herein, “essentially serum free” refers to a medium that does not contain serum or serum replacement, or that contains essentially no or very little serum or serum replacement. As used herein, “essentially” means that a de minimus or reduced amount of a component, such as serum, may be present that does not eliminate the improved bioactive neural cell culturing capacity of the medium or environment. For example, essentially serum free medium or environment can contain less than 10, 9, 8, 7, 6, 5, 4, 3, 2, or 1% serum or serum replacement wherein the presently improved bioactive neural cell culturing capacity of the medium or environment is still observed.

A differentiating medium or environment may be utilized to partially, terminally, or reversibly differentiate the neural cells of the present invention, either prior to or after contacting the neural cells with MEDII conditioned medium. In accordance with the invention, the differentiating medium may contain a variety of components including, KODMEM medium (Knockout Dulbecco's Modified Eagle's Medium), FBS, FGF2 and hLIF. In another embodiment, the differentiating medium may contain a DMEM/F12 medium, KSR, and FGF2. The conditioned medium may also contain supplements such as L-Glutamine, NEAA (non-essential amino acids), P/S (penicillin/streptomycin) and β-mercaptoethanol (β-ME). Preferably, the KSR is added at approximately between a 1-30% concentration, and more preferably at approximately a 5% concentration. In one embodiment, the differentiating medium is a DMEM/F12 medium. In one embodiment of the present invention, the differentiating medium comprises a base salt solution. Preferably, the base salt solution is selected from the group consisting of DMEM, GMEM, and mixtures thereof.

It is to be understood that the step of contacting the cell with the MEDII conditioned medium includes the use of a “normal” or “other” medium supplemented with a MEDII conditioned medium. The “normal” or “other” medium, such as a normal primate ES medium, can be supplemented with a MEDII conditioned medium at any concentration, but it is preferred that the “normal” or “other” medium is supplemented at between approximately 10-75%, more preferably between approximately 40-60% and most preferably approximately 50% MEDII conditioned medium.

As used herein, the term “cell differentiation environment” refers to a cell culture condition wherein the pluripotent cells are induced to differentiate into neural cells, or are induced to become a human cell culture enriched in neural cells. Preferably, the neural cell lineage induced by the growth factor will be homogeneous in nature. The term “homogeneous,” refers to a population that contains more than 50%, 60%, 70%, 80%, 85%, 86%, 87%, 88%, 89%, 90%, 91%, 92%, 93%, 94%, 95%, 96%, 97%, 98%, or 99% of the desired neural cell lineage.

In other embodiments, the cell differentiation environment comprises plating the neural cells in an adherent culture. As used herein, the terms “plated” and “plating” refer to any process that allows a cell to be grown in adherent culture. As used herein, the term “adherent culture “refers to a cell culture system whereby cells are cultured on a solid surface, which may in turn be coated with a solid substrate that may in turn be coated with another surface coat of a substrate, such as those listed below, or any other chemical or biological material that allows the neural cells to proliferate or be stabilized in culture. The cells may or may not tightly adhere to the solid surface or to the substrate. The substrate for the adherent culture may further comprise any one or combination of polyornithine, laminin, poly-lysine, purified collagen, gelatin, extracellular matrix, fibronectin, tenascin, vitronectin, poly glycolytic acid (PGA), poly lactic acid (PLA), poly lactic-glycolic acid (PLGA) and feeder cell layers such as, but not limited to, primary astrocytes, astrocyte cell lines, glial cell lines, stromal cells, primary fibroblasts or fibroblast cells lines. In addition, primary astrocyte/glial cells or cell lines derived from particular regions of the developing or adult brain or spinal cord including but not limited to olfactory bulb, neocortex, hippocampus, basal telencephalon/striatum, midbrain/mesencephalon, substantia nigra, cerebellum or hindbrain may be used to enhance the development of specific neural cell sub-lineages and neural phenotypes. Furthermore, the substrate for the adherent culture may comprise the extracellular matrix laid down by a feeder cell layer, or laid down by the pluripotent human cell or cell culture.

The methods of-the present invention contemplate that cells may be cultured with a feeder cell. As used herein, a “feeder cell” is a cell that is co-cultured with a pluripotent cell and maintains the pluripotent cell in an undifferentiated or partially differentiated state. In one embodiment of the above method, the conditioned medium is obtained from a feeder cell that maintains the human pluripotent cell in an undifferentiated or partially differentiated state. In addition, as used herein, the term “feeder cell” is used to define a cell that is co-cultured with a neural cell and stabilizes the neural cell in a partially differentiated state. As an example, the neural cell of the present invention can be cultured with a MEDII producing feeder cell so that the neural cell is maintained in a partially differentiated, neural progenitor, state. Any and all factors produced by a feeder cell that allow a pluripotent cell to be maintained in an undifferentiated or partially differentiated state, or that stabilizes a neural cell in a partially differentiated state can be isolated and characterized using methods routine to those of skill in the art. These factors may be used in lieu of a feeder cell layer, or may be used to supplement a feeder cell layer.

The neural cells produced using the methods of the present invention have a variety of uses. In particular, the neural cells can be used as a source of nuclear material for nuclear transfer techniques and used to produce cells, tissues or components of organs for transplant. The neural cells of the present invention can also be used in human cell therapy or human gene therapy to treat neuronal diseases such as Parkinson's disease, Huntington's disease, lysosomal storage diseases, multiple sclerosis, memory and behavioral disorders, Alzheimer's disease and macular degeneration. Other pathological conditions including stroke and spinal cord injury can be treated using the neural cells of the present invention. The neural cells produced using the methods of the present invention can be distinguished from neurospheres in that the cells of the current invention are an adherent culture whereas neurospheres are a suspension culture. In addition the neural cells produced by the methods of the current invention appeared to differ in morphology to the neurectoderm cells produced in accordance with WO 01/51611.

Using the methods of the present invention it was possible to produce a neural progenitor cell capable of differentiating into a neural cell population where at least 50% of the cells were TH positive. It was also possible to produce a neural progenitor cell that is capable of differentiating into glial cells, and other types of neural cells.

Throughout this application, various publications are referenced. The disclosures of all of these publications and those references cited within those publications in their entireties are hereby incorporated by reference into this application in order to more fully describe the state of the art to which this invention pertains.

### EXAMPLES

### Example 1

Production of MEDII Conditioned Media

A MEDII conditioned medium was produced as follows. HepG2 cells (Knowles et al., 1980 Nature 288:615-618; ATCC HB-8065) were maintained in culture in DMEM and passaged at confluence. To condition medium (then called MEDII), HepG2 cells were seeded into DMEM at a density of 5×10⁴ cells/cm². Medium was collected after 4-5 days, sterilised by filtration through a 0.22 μm membrane and supplemented with 0.1 mM β-ME, 1,000 IU/ml hLIF and 4 ng/ml FGF2 before use. MEDII was stored at 4° C. for 1-2 weeks or at −20° C. for up to 6 months without apparent loss of activity. A further explanation of MEDII conditioned media can be found in International Application No. WO 99/53021.

The bioactive components of MEDII can be isolated and characterized using techniques routine to those of ordinary skill in the art. Non-limiting examples of such isolation and characterization can be found within International Application No. WO 99/53021, herein incorporated by reference in its entirety.

### Example 2

Stabilization of Neural Cells Derived from HES Cells Cultured with a Stromal Cell Line by Further Contacting the Neural Cells with MEDII Conditioned Medium

The mouse stromal cell line PA6 was used as a substrate for the culture and differentiation of human ES cells. The human ES cell line BG01 was manually passaged onto a monolayer of stromal PA6 cells not later than three days after the stromal cells were passaged. The medium used was HES medium, comprising DMEM/F12, 15% FBS, 5% KSR, 1 mM glutamine, 1× NEAA, penicillin/streptomycin, beta mercaptoethanol, and FGF2. The cells were allowed to differentiate for 33 days in total. For the first 16 days, the medium was changed every two days without passaging the cells. The medium of the ES cultures that contained HES medium at 16 days of culture was replaced in its entirety with essentially serum free DMEM/F12, and the cultures were allowed to differentiate another 17 days.

In these conditions, the human ES cells formed colonies with columnar cell morphology, termed rosette structures. At day 33, the rosette structures were manually picked up with Pasteur pipettes and plated onto polyornithine-laminin coated dishes. These cells were purified manually for morphological evaluation of neural progenitors (according to the morphology of mouse neural progenitors) and were cultured in essentially serum free medium comprising 50% MEDII conditioned DMEM/F12 medium, 50% DMEM/F12, 1×N2, and 4 ng/ml FGF2 for over twelve months.

During the first several passages, a mixture of cell types with different morphologies was observed. From every other passage, cells were selected that had the rosette morphology, such that cells with the non-rosette morphology were left behind.

Characterization of the passaged cells by immunostaining demonstrated that the cells with columnar rosette morphology were positive for early neuronal markers. The cells were positive for the intermediate filament protein nestin and for the neural RNA binding protein Musashi-1, which has been used to identify neuroepithelial stem cells. These cells did not express markers of differentiated neural cell types such as b-Tubulin type III, MAP-2ab, or GFAP. These cultures were therefore enriched for neural progenitor cells.

The enriched cultures of neural progenitor cells were capable of propagation on a variety of substrates in adherent culture on coated culture dishes. Efficiency of culturing was determined on plastic and glass dishes where the substrate was selected from the group consisting of no substrate, gelatin, poly-L-ornithine, and laminin. The adherence and growth of the neural precursors was most effective in the presence of combination of poly-L-ornithine and laminin on plastic dishes. Coating the dishes with gelatin, poly-L-ornithine or with laminin alone resulted in a lower efficiency of adherence.

When cultured in DMEM/F12 medium plus 4 ng/ml bFGF and 1×N2 supplements, the neural progenitor cells failed to proliferate after 12 days in culture and extensive cell death was observed. However, when this same medium was supplemented with MEDII, the neural progenitor cells proliferated indefinitely, still maintaining the columnar, rosette morphology. The neural progenitor cells cultured in MEDII supplemented DMEM/F12 continues to proliferate for 12 months when passaged every 5 to 7 days.

It was also determined that the addition of human LIF into the culture medium did not appear to affect the differentiation of the neural progenitor cells. The neural progenitor cells were cultured in the presence or absence of 1000 U/ml hLIF for 5 passages and were further analyzed for the expression of nestin and Musashi-1. Cells cultured in the presence and absence of hLIF had similar rate of nestin+and Musashi-1+ expression. Neither culture expressed non-neural cell markers. The cells were negative for alpha-fetoprotein, muscle actin, and Cytokeratin-8.

The differentiation of the neural precursor cells grown on poly-L-ornithine-laminin in the presence of 50% MEDII/50% F12, supplemented with N2 and FGF2 was evaluated. The cells were allowed to differentiate by replacing the MEDII containing medium with DMEM/F12 medium containing approximately 5% FBS. After 3 days in culture, the medium was changed to Neurobasal medium (Invitrogen) containing 1×N2, but with essentially no FGF2. Over the next 5-7 days, extensive neuronal differentiation occurred, as determined by the morphology of the cells. Immunostaining showed that the neural precursor cells gave rise to all major neural phenotypes, including but not limited to dopaminergic neurons, gabanergic neurons, and glial cells. After 5-7 days of culture, many of the neurons formed expressed markers that are characteristic of immature neurons, such as βIII-Tubulin and microtubule associated protein MAP2abc. Furthermore, when neuronal cultures were incubated in Neurobasal medium for 10 or more days, the differentiated neural progenitor cells expressed markers of mature neurons such as MAP2ab, and 200 kDa neurofilament NF200, tyrosine hydroxylase (TH), DAT and VMAT2. A high proportion of the cells expressed TH. In addition, in certain cells, coexpression of TH with bTubulin III and MAP2ab was observed. In other cells, co-expression of TH with DAT and VMAT2 was observed.

These progenitor cultures were frozen and successfully thawed, and the cells maintained the characteristics described above. Thus, these cell lines were capable of proliferating indefinitely in vitro without senescence, and more importantly, the cell lines were stabilized due to co-culture with MEDII conditioned medium and/or stromal cells. These stabilized neural progenitor cells were capable of differentiating into multiple neural cell types under defined conditions.

### Example 3

Stabilization of Neural Cells Derived from HES Cells Cultured with MEFs by Further Contacting the Neural Cells with MEDII Conditioned Medium

To confirm that culture with PA6 stromal cells was not necessary for the formation of a neural progenitor cell that can be stabilized by MEDII conditioned medium, Example 3 was repeated, except the mouse embryonic fibroblasts (MEFs) were used as the feeder cell layer instead of PA6 cells. The media used and the timing of media changes were approximately the same as in Example 3.

The neural progenitor cells obtained from co-culture with the MEFs were cultured with MEDII supplemented DMEM/F12/bFGF/N2 medium. These cells continued to proliferate in culture for greater than 12 months when passaged every 5-7 days. When compared to cells originally cultured with PA6 cells, these neural progenitor cells had a similar rate of nestin and Musashi-1 expression, and the cells did not express non-neural cell markers.

The in vitro differentiation of these neural progenitor cells was examined as in Example 3. The neural progenitors gave rise to the same neural types as the cells cultured with PA6 cells. No difference in the proportions of neurons formed was observed between the two types of neural progenitors.

To evaluate the stromal cell-derived inducing activity (SDIA) that promotes neural differentiation to dopaminergic neurons, the neural progenitors derived on PA6 cells were compared to neural progenitors derived on MEFs in their ability to express TH. Both cell lines showed similar differentiation potential to mature neurons, and in particular, to TH positive cells. To estimate the efficiency of dopaminergic neuron formation, the proportion of differentiated cells positive for βIII-Tubulin, nestin and TH were determined. Approximately 78.5% of the differentiated cells were positive for TH, and approximately 93.5% were positive for βIII-Tubulin. The proportions of the differentiated cells generated from the neural progenitors derived on MEFs and derived on PA6 cells were similar.

These results confirmed that neural progenitors were stabilized in culture due to the culture with the MEDII containing medium, and that the neural inducing capabilities of the PA6 stromal cell line was not critical nor necessary for the effects noted herein.

1. A method of stabilizing a neural cell comprising:
(a) providing a pluripotent mammalian cell;
(b) culturing the pluripotent mammalian cell to produce a neural cell; and
(c) contacting the neural cell with a MEDII conditioned medium for greater than 2 passages to thereby stabilize the neural cell.

2. The method of claim 1, whereby step (b) occurs in the presence of a feeder cell layer.

3. The method of claim 1 wherein step (b) comprises the use of a medium comprising a compound selected from the group consisting of KSR, GMEM, HES medium, or any combination thereof.

4. The method of claim 1, further comprising an additional step of contacting the cells with a differentiating medium prior to contact with the MEDII conditioned medium.

5. The method of claim 4, wherein the differentiating medium is essentially serum free.

6. The method of claim 4 where the differentiating medium further comprises a base salt solution.

7. The method of claim 6 where the base salt solution selected from the group consisting of DMEM, GMEM or any combination thereof.

8. The method of claim 4 wherein the differentiating medium further comprises supplements selected from the group consisting of N2, FGF2, or any combination thereof.

9. The method of claim 1, wherein the MEDII conditioned medium is essentially serum free.

10. The method of claim 1, wherein the mammalian pluripotent cell is selected from the group consisting of an embryonic stem cell, an ICM/epiblast cell, a primitive ectoderm cell, a primordial germ cell, and a teratocarcinoma cell.

11. The method of claim 10, wherein the mammalian pluripotent cell is a human embryonic stem cell.

12. The method of claim 2, wherein the feeder cell layer comprises a stromal cell.

13. The method of claim 12, wherein the stromal cell is a murine stromal cell.

14. The method of claim 13, wherein the stromal cell is a PA6 cell.

15. The method of claim 12, wherein the stromal cell is human stromal cell.

16. The method of claim 1, wherein the MEDII conditioned medium is a HepG2 conditioned medium.

17. The method of claim 1, wherein the MEDII conditioned medium comprises a biologically active component selected from the group consisting of:
(a) a large molecular weight extracellular matrix protein;
(b) a low molecular weight component comprising proline;
(c) a biologically active fragment of any of the proteins or components described in a) or b);
(d) an analog of any of the proteins or components described in a) or b);
(e) a neural inducing factor; and
(f) any combination thereof.

18. The method of claim 1, wherein the MEDII conditioned medium comprises a large molecular weight extracellular matrix protein.

19. The method of claim 1 wherein the neural cell is plated prior to contact with the MEDII conditioned medium.

20. The method of claim 19, wherein the neural cell is plated with a feeder cell layer prior to contact with the MEDII conditioned medium.

21. The method of claim 19, wherein the neural cell is plated on a substrate prior to contact with the MEDII conditioned medium.

22. The method of claim 1, further comprising the step of isolating the neural cell after step (b) prior to contacting the neural cell with the MEDII conditioned medium.

23. The method of claim 22, wherein isolating the neural cell comprises manually selecting the neural cell.

24. The method of claim 1, further comprising the subsequent step of differentiating the stabilized neural cell to produce a differentiated neural cell.

25. The method of claim 24, wherein the differentiated neural cell is TH positive, and expresses DAT and V-MAT.

26. The method of claim 25, wherein a population of differentiated neural cells is produced, and at least 50% of the population of differentiated neural cells is TH positive.

27. The method of claim 24, wherein the differentiated neural cell expresses glutamate decarboxylase.

28. The method of claim 24, wherein the differentiated neural cell expresses GFAP.

29. The method of any one of claims 1-28, wherein the neural cell is a neural progenitor cell.

30. A method of stabilizing a neural cell comprising:
(a) providing a mammalian neural cell; and
(b) contacting the neural cell with a MEDII conditioned medium for greater than 2 passages to thereby stabilize the neural cell.

31. The method of claim 30, wherein the neural cell is plated prior to contact with the MEDII conditioned medium.

32. The method of claim 31, wherein the neural cell is plated with a feeder cell layer prior to contact with the MEDII conditioned medium.

33. The method of claim 32, wherein the feeder cell layer comprises a stromal cell.

34. The method of claim 33, wherein the stromal cell is human stromal cell.

35. The method of claim 31, wherein the neural cell is plated on a substrate prior to contact with the MEDII conditioned medium.

36. The method of claim 30, wherein the neural cell is a neural progenitor cell.

37. The method of claim 30, wherein the mammalian neural cell is a human neural cell.

38. The method of claim 30, wherein the MEDII conditioned medium is a HepG2 conditioned medium.

39. The method of claim 30, wherein the MEDII conditioned medium is essentially serum free.

40. The method of claim 30, wherein the MEDII conditioned medium comprises at least a biologically active component selected from the group consisting of:
(a) a large molecular weight extracellular matrix protein;
(b) a low molecular weight component comprising proline;
(c) a biologically active fragment of any of the proteins or components described in a) or b);
(d) an analog of any of the proteins or components described in a) or b);
(e) a neural inducing factor; and
(f) any combination thereof.

41. The method of claim 30, further comprising the additional step of differentiating the stabilized neural cell to produce a differentiated neural cell.

42. The method of claim 41, wherein the differentiated neural cell is TH positive, and expresses DAT and V-MAT.

43. The method of claim 42, wherein a population of differentiated neural cells is produced, and at least 50% of the population of differentiated neural cells are TH positive.

44. The method of claim 41, wherein the differentiated neural cell expresses glutamate decarboxylase.

45. The method of claim 41, wherein the differentiated neural cell expresses GFAP.

46. A composition comprising an isolated neural cell, wherein the cell expresses nestin, wherein the cell has been stabilized by contact with MEDII conditioned medium for greater than 2 passages, and wherein the cell can differentiate into more than one type of further differentiated neural cell.

47. The composition of claim 46, wherein the cell has been stabilized by contact with MEDII conditioned medium for greater than 5 passages.

48. The composition of claim 46, wherein the cell has been stabilized by contact with MEDII conditioned medium for greater than 10 passages.

49. The composition of claim 46, wherein the cell has been stabilized by contact with MEDII conditioned medium for greater than 20 passages.

50. The composition of claim 46, wherein the cell has been stabilized by contact with MEDII conditioned medium for greater than 30 passages.

51. The composition of claim 46, wherein the cell has been stabilized by contact with MEDII conditioned medium for more than one year.

52. The composition of claim 46, wherein the neural cell is a neural progenitor cell.

53. The composition of claim 46, wherein the MEDII conditioned medium is a HepG2 conditioned medium.

54. The composition of claim 46, wherein the MEDII conditioned medium is essentially serum free.

55. The composition of claim 46, wherein the MEDII conditioned medium comprises at least a biologically active component selected from the group consisting of:
(a) a large molecular weight extracellular matrix protein;
(b) a low molecular weight component comprising proline;
(c) a biologically active fragment of any of the proteins or components described in a) or b);
(d) an analog of any of the proteins or components described in a) or b);
(e) a neural inducing factor; and
(f) any combination thereof.


SEQUENCE LISTING (5 sequences)

SEQ ID NO: 1 - protein, 4 aa, Artificial Sequence
  Description of Artificial Sequence Synthetic peptide
GPRP

SEQ ID NO: 2 - protein, 4 aa, Artificial Sequence
  Description of Artificial Sequence Synthetic peptide
GPGG

SEQ ID NO: 3 - protein, 4 aa, Artificial Sequence
  Description of Artificial Sequence Synthetic peptide
VAPG

SEQ ID NO: 4 - protein, 4 aa, Artificial Sequence
  Description of Artificial Sequence Synthetic peptide
RPKP

SEQ ID NO: 5 - protein, 11 aa, Artificial Sequence
  Description of Artificial Sequence Synthetic peptide
RPKPQQFFGLM